(19) United States
(12) Patent Application Publication
(10) Pub. No.: US 2026/0108153 A1
(43) Pub. Date: Apr. 23, 2026

(54) A Computer Implemented Method for Obtaining Real-Time Maps of Physical Quantities Characterizing Complex Uncertain Systems from Scarce and Indirect Measurements

(71) Applicants: Fondazione Links - Leading Innovation & Knowledge for Society, Torino (IT); Politecnico Di Torino, Torino (IT)

(72) Inventors: Rossella Gaffoglio, Santo Stefano Belbo (IT); Giorgio Giordanengo, Robilante (IT); Marco Righero, Torino (IT); Giuseppe Vecchi, Leini (IT)

(21) Appl. No.: 19/117,877

(22) PCT Filed: Oct. 2, 2023
(86) PCT No.: PCT/IB2023/059859

§ 371 Date: Apr. 2, 2025

(30) Foreign Application Priority Data
Oct. 3, 2022 (IT) 102022000020310

(51) Int. Cl. A61B 5/00; A61B 5/055; A61B 6/03; A61B 18/00; A61N 5/02

(52) U.S. Cl. CPC A61B 5/0036; A61B 5/055; A61B 6/032; A61N 5/025; A61B 2018/00791

(57) ABSTRACT

A computer implemented method for obtaining a real-time map, h(x), of a distribution in a D-dimensional space of a physical quantity, ƒ(x), describing a true phenomenon characterizing a complex uncertain system, wherein said physical quantity, ƒ(x), is inaccessible to direct measurements. The method can be utilized to obtain real time maps characterizing complex uncertain systems from scarce and indirect measurements, using non-invasive techniques.

The present invention relates to a computer implemented method for obtaining real-time maps of physical quantities characterizing complex uncertain systems from scarce and indirect measurements.

The attempt to reliably reconstruct the physical properties of a system, from scarce and indirect observations of the system itself, represents a crucial and open problem in many areas of investigation, e.g., medical imaging, epidemiology, astronomy, geophysics.

Different approaches can be found in the literature, where this problem is addressed through inverse methods based on Bayesian inference and neural networks (see patent application no. WO2015016990A1; G. Wang, J. C. Ye, and B. De Man, “Deep learning for tomographic image reconstruction”, Nat. Mach. Intell., vol. 2, pp. 737-48, 2020).

In particular, a first area of investigation is the temperature monitoring in microwave cancer hyperthermia.

Microwave hyperthermia is a type of cancer treatment in which tumor cells are selectively exposed to a supra-physiological temperature (42-44° C.) by means of proper antenna systems to negatively impact cancer growth (H. H. Kampinga, “Cell biological effects of hyperthermia alone or combined with radiation or drugs: a short introduction to newcomers in the field”, Int. J. Hyperthermia, vol. 22, no. 3, pp. 191-6, 2006).

In combination with radiotherapy and/or chemotherapy, hyperthermia has been proven to be beneficial for treatment outcome for a large variety of tumors (N. R. Datta et al., “Local hyperthermia combined with radiotherapy and/or chemotherapy: Recent advances and promises for the future”, Cancer Treat. Rev., vol. 41, no. 9, pp. 742-53, 2015).

Temperature control is the primary technological challenge in cancer hyperthermia, especially for deep-seated tumors. In this context, treatment planning is fundamental to optimally set the antenna feedings of the applicator to maximize the temperature increase in the tumor, minimizing the risk of overheating in the surrounding healthy tissues. This is performed by means of proper numerical solvers, where both the patient's phantom (derived from CT and MRI scans) and the antenna applicator are modelled, and an optimization of the antenna feedings is implemented to focus the specific absorption rate (SAR)—or directly the temperature—on the tumor target (M. M. Paulides et al., “Simulation techniques in hyperthermia treatment planning”, Int. J. Hyperthermia, vol. 29, no. 4, pp. 346-57, 2013).

One of the greatest limitations of simulation-based planning is the uncertainty characterizing the dielectric and (in particular) the thermal parameters of the different tissues, which must be assigned to the segmented phantom before solving Maxwell's and bioheat equations. This uncertainty could lead to unreliable temperature predictions in the region of interest (ROI), making essential the acquisition of direct measurements during treatment. Intraluminal thermometry, i.e., catheters placed in the body cavities, often proves useless due to the distance from the tumor site, and the high degree of uncertainty introduced by physiology (e.g., breathing, swallowing and variable tissue contact in the oral cavity for tumors in the head and neck region). Therefore, in the current clinical practice, invasive interstitial catheters are necessary to obtain some reliable temperature measurements (M. M. Paulides, G. M. Verduijn, and N. Van Holthe, “Status quo and directions in deep head and neck hyperthermia”, Radiat. Oncol., vol. 11, no. 21, 2016). Besides causing great discomfort to the patient, these local measurements provide very limited spatial information.

Different techniques for non-invasive thermometry during hyperthermia have been investigated from the very beginning, but an adequately reliable method suitable for a widespread use in the clinic is still lacking.

Studies on the use of microwave radiometry as non-invasive method of measuring temperature date back to 1974-75 (A. H. Barret, and P. C. Myers, “Microwave thermography: a method of detecting subsurface thermal patterns”, Bibl. Radiol., vol. 6, pp. 45-56, 1975). This technique requires the definition of mathematical (inverse) problems to retrieve the temperature information of subcutaneous tissues from the measurement of the emitted black body radiation. The extremely weak signal level, the sparsity of data and the noise contamination make this technique really challenge and unable to provide deep temperature estimations. Moreover, concurrent use with hyperthermia poses further technical problems, due to the presence of the radiating applicator (S. Jacobsen and P. R. Stauffer, “Multifrequency Radiometric Determination of Temperature Profiles in a Lossy Homogeneous Phantom Using a Dual-Mode Antenna With Integral Water Bolus”, IEEE Trans. Microw. Theory Techn., vol. 50, no. 7, 2002).

Another method to gather indirect non-invasive information on temperature during thermal therapies is radio frequency (RF) tomography (microwave or impedance tomography) (M. Haynes, J. Stang, and M. Moghaddam, “Real-time microwave imaging of differential temperature for thermal therapy monitoring”, IEEE Trans. Biomed. Eng., vol. 61, no. 6, 2014; M. Bevacqua et al., “A Method for Effective Permittivity and Conductivity Mapping of Biological Scenarios via Segmented Contrast Source Inversion”, Prog. Electromagn. Res., vol. 164, 2019), which derives temperature information from the change in the dielectric properties of body tissues.

All these techniques for non-invasive temperature control are still under development, and a use of these methods in the clinical practice remains a distant goal. Therefore, at present, thermometric data from radiometry and RF tomography are to be considered a scarce, indirect, and highly uncertain source of information.

Probes for estimating the deep body temperature from the skin surface using heat-flux measurements have also been implemented (patent application no. WO2011126543A1; K.-I. Kitamura et al., “Development of a new method for the non-invasive measurement of deep body temperature without a heater”, Med. Eng. Phys., vol. 32, 2010), and could be considered as other means to provide indirect non-invasive temperature information during thermal therapies. In the recent years, research has been directed towards the possibility to control the temperature in the patient during hyperthermia treatments using magnetic resonance (MR) thermometry (G. C. van Rhoon, and P. Wust, “Introduction: non-invasive thermometry for thermotherapy”, Int. J. Hyperthermia, vol. 21, no. 6, pp. 489-95, 2005): although promising, temperature monitoring with MR could be affected by inhomogeneities in the magnetic flux density and motion artifacts. Moreover, this technique requires the therapy to take place inside a MR scanner, with a suitable MR-compatible antenna set-up, limiting the possibility of doing this type of temperature monitoring to specifically organized clinical centres.

A second area of investigation is the temperature monitoring in electronic devices.

One of the challenges in the design of electronic devices is the appropriate handling of thermal load. This issue affects both the design of integrated circuits (ICs) and the spatial arrangement inside the housing. The main difficulty is related to the measurement of temperature at critical points during runtime.

For what concerns the ICs, one wants to estimate the chip level thermal profile from runtime temperature sensor readings and faces the problem of having only few sensors that are not even placed in the points of interest. One solution to that is to assume the power density to be stochastic with known mean and variance, and then estimate a-posteriori mean and variance of temperature on the whole chip from readings of the temperature at some fixed locations on the chip (Y. Zhang et al. “Chip Level Thermal Profile Estimation Using On-chip Temperature Sensors”, 2008 IEEE Int. Conf. on Computer Design, 2008). Efficient methods to derive the thermal profile from the power density profile are needed to use this approach (Y. Zhan and S. S. Sapatnekar, “High-Efficiency Green Function-Based Thermal Simulation Algorithms”, IEEE Trans. On CAD of Int. Circ. and Syst., vol. 26, no. 9, 2007).

A black-box model based on a neural-network trained to correlate the readings of some performance counters—such as the number of executed instructions, CPU cycles (frequency), executed branches—to the temperature profile on the chip is described in M. Rapp, O. Elfatairy, M. Wolf, J. Henkel and H. Amrouch, “Towards NN-based Online Estimation of the Full-Chip Temperature and the Rate of Temperature Change”, 2020 ACM/IEEE 2nd Workshop on Machine Learning for CAD (MLCAD), 2020.

For what concerns the whole housing, one is interested in knowing the temperature on the whole housing of the device for safety reasons, as many devices are intended to be used in direct physical contact with a user. Common approaches are based on processing of available data and on use of regression analysis. To compensate the fact that sensors are rarely placed at the most convenient location to record temperature and that the locations of the hottest points may vary at runtime—for instance if a DVD reader is inserted or a PCI card is added—(see U.S. Pat. No. 8,762,097B2) the use of virtual sensors has been proposed. These are mathematical models, built prior to selling the devices to the general public, that correlate various input sources, such as system power sensors, physical temperature sensors, and/or system configuration information (e.g., cooling system status, such as fan speed for each fan, etc.), to the temperature measured at the location of interest under different working conditions. A similar idea is described in U.S. Ser. No. 10/488,873B2, where the temperature at hotpots on the surface of an electronic device is correlated with the temperature of an internal part, whose reading is then used to estimate the temperature on the surface and perform a control procedure to lower it, when it exceeds a threshold value.

To face the issue that available data do not satisfy common assumptions for the validity of regression analysis (e.g., data are correlated in time, but regression analysis assumes they are not), one may split the response into a steady-state (that is not correlated in time) and transient, and estimate the latter using a filtering algorithm based on considering three frequencies in the response (see U.S. Pat. No. 9,546,914B2).

In any case, none of the above mentioned approaches allows to satisfactorily measure the thermal load of electronic devices at critical points during runtime.

A third area of investigation is the monitoring of the spreading of a malicious agent in a network of agents.

Starting from the studies of Bernoulli and Snow in the XVIII and XIX centuries, respectively, (D. Bernoulli, “Essai d'une nouvelle analyse de la mortalité causée par la petite vérole at des advantages de l'inoculation pour la prévenir”, Mem. de Math. E de Phys, Acad. R de Sc. 1760; J. Snow, “On the mode of communication of cholera”, John Churchill, London, 1855) scientists have now many mathematical tools to model and analyse the diffusion processes in an ensemble of interacting agents (H. Hethcote, “The mathematics of infectious diseases”, SIAM Rev., vol. 42, no, 2, 2000).

These tools are applied to try to answer different questions, as pinning down the source of the infection (D. Shah and T. Zaman, “Detecting sources of computer viruses in networks: theory and experiment”, ACM SIGMETRICS Perf. Eval. Review, vol. 3, no. 1, 2010; D. Shah and T. Zaman, “Rumors in a Network: Who's the Culprit?,” IEEE Trans. on Inf. Th., vol. 57, no. 8, 2011; C. H. Comin and L. da Fontoura Costa, “Identifying the starting point of a spreading process in complex networks”, Phys. Rev. E, vol. 84, 2011; N. Antulov-Fantulin et al., “Statistical Inference Framework for Source Detection of Contagion Processes on Arbitrary Network Structures,” 2014 IEEE Eighth International Conference on Self-Adaptive and Self-Organizing Systems Workshops, 2014; F. Altarelli et al. “Bayesian inference of epidemics on networks via belief propagation”, Phys. Rev. Lett., vol. 112, no. 11, 2014), analysing the cost-benefit trade-off of different policies to curb the diffusion (A. Baker et al, “Epidemic mitigation by statistical inference from contact tracing data”, PNAS, vol. 118, no. 32, 2021), and devising optimal immunization strategies (F. Altarelli et al. “Containing Epidemic Outbreaks by Message-Passing Techniques”, Phys. Rev. X vol. 4, no. 2, 2014).

At the core of these tools there are models of the diffusion process, from simpler (R. Ross, “An application of the theory of probabilities to the study of a priori pathometry.—Part I”, Proc. R. Soc. Lond. A, vol. 92, no. 638, 1916; R. Ross and H. Hudson “An application of the theory of probabilities to the study of a priori pathometry.—Part II”, Proc. R. Soc. Lond. A, vol. 93, no 650, 1917; R. Ross and H. Hudson, “An application of the theory of probabilities to the study of a priori pathometry.—Part III”, Proc. R. Soc. Lond. A, vol. 93, no. 65, 1917; W. O. Kermack and A. G. McKendrick, “A contribution to the mathematical theory of epidemics”, Proc. R. Soc. Lond. A, vol. 115, no. 772, 1927; D. Kendall, “Deterministic and stochastic epidemics in closed populations”, in J. Neymann (ed.) Contributions to Biology and Problems of Health, vol. 4., University of California Press, Berkeley, 2020) to more complex ones (R. Hinch. et al., “OpenABM-Covid19—An agent-based model for non-pharmaceutical interventions against COVID-19 including contact tracing”, PLOS Comp. Biology, vol. 17, no. 7, 2021), and a description of the network of interactions. Both aspects are severely affected by uncertainties and incompleteness of data (M. E. J. Newman, “Network structure from rich but noisy data”, Nature Phys., vol. 14, 2018).

It is therefore an object of the present invention to provide a computer implemented method for obtaining real-time maps of physical quantities characterizing complex uncertain systems from scarce and indirect measurements, wherein said real-time maps are complete, accurate, physically sound, and highly reliable.

It is a further object of the present invention to provide a computer implemented method for obtaining real-time maps of physical quantities characterizing complex uncertain systems from scarce and indirect measurements, wherein said real-time maps can be obtained for any complex uncertain system.

It is a further object of the present invention to provide a computer implemented method for obtaining real-time maps of physical quantities characterizing complex uncertain systems from scarce and indirect measurements, wherein said real-time maps can be obtained with non-invasive techniques.

The proposed invention concerns the implementation of a technology to determine in real time an accurate map of a physical quantity using measured data that may be scarce and indirectly correlated to the quantity of interest.

By scarce set of measurement data, it is meant a set of measurements data which are not able to characterize per se a phenomenon.

The physical quantity of interest may characterize a complex phenomenon whose knowledge is affected by uncertainty.

The proposed technique provides a reliable representation of the phenomenon by matching the few available indirect measurements to a library of different models of the system under investigation.

Multiphysics simulations of imperfect copies of the system may be used to populate such library, where the copies have variations purposely added to account for all the uncertainties affecting the modelling.

The representation obtained at the end of the matching process is termed hyper-physics framework.

The presented method can find application in all fields of engineering, physics, or social sciences where the real-time monitoring of a physical quantity describing a complex system is required and only few indirect measurements are available.

The fundamental aim of the method here presented is to provide an accurate and reliable assessment of a physical quantity concerning a complex and uncertain system, which cannot be measured directly, due to safety reasons, impracticability, or budget issues. Sources of the physical quantity of interest are difficult to reach, whereas some of the effects can be visible. If, on one hand, direct measurements are not available, on the other hand, the numerical simulation of the system cannot provide reliable results per se, due to the unavoidable uncertainty characterizing some of the model parameters, and even some of the model equations.

More in particular, the first step of the proposed method consists in creating a large set of simulations of purposely mutated replicas of the system, called augmented model, where the different parameters are changed to describe the variety of states in which the system might exist. Upon appropriate processing, this enlarged set of simulations constitutes an a-priori source of information, that will collapse towards a reliable representation of the system via a set of measurements, even if indirect and inaccurate. In other words, all real-time data acquisition can be incorporated into the simulative framework of the augmented model, and the actual performance of the system can be conveniently recovered at any time by means of a “model-based interpolation”.

In the following, the proposed method will be described first as a general technique that can be applied to any uncertain system, using indirect, scarce measurements of the involved physical quantities.

Then, the description will be focused on the specific example of temperature monitoring in microwave cancer hyperthermia. In this context, the uncertain system is the human body under treatment, the augmented model consists of a library of patient-specific simulations of purposely mutated replicas of the patient, and the physical quantity of interest is the temperature in the whole region of interest. By applying the proposed method, the inherent information included in the augmented model will be sufficient to provide reliable temperature maps of the patient, only using real-time data acquired with non-invasive thermographic techniques.

Finally, two other applications of the method according to the invention presented here concern the temperature monitoring in electronic devices and the monitoring of the spreading in a network of agents that communicate (agents may be people or computers and the malicious agent may be a pathogen or a malicious software).

In one embodiment of the present invention, it is described a computer implemented method for obtaining a real-time map, h(x), of a distribution in a D-dimensional space of a physical quantity, ƒ(x), describing a true phenomenon characterizing a complex uncertain system, wherein the physical quantity, ƒ(x), is inaccessible to direct measurements; the method comprises the steps of: describing the complex uncertain system by means of a mathematical model for simulating a spatial distribution, \hat{ƒ}(x; s), of the physical quantity, ƒ(x), using a set of parameters (s); building a library of approximations, \hat{ƒ}(x; sₙ), n=1, . . . , N, of the physical quantity, ƒ(x), by running a plurality of simulations through assignment of specific sets of values, sₙ, to the set of parameters, s; calculating said real-time map, h(x), representing a real-time approximation of the physical quantity, ƒ(x), in a form

$$h ( x ) = {\sum }_{n = 1}^{N} {a}_{n} \hat{f} ( x ; {s}_{n} ) ,$$

wherein aₙ, n=1, . . . , N, are expansion coefficients which are determined by solving a system of L equations with N unknowns so as to minimize the magnitude of the discrepancies | [symbol] |, where [symbol] =1, . . . , L, and the discrepancies are

$${v}_{ℓ} = {𝒜}_{ℓ} ( f ( x ) , {x}_{ℓ} ) - {\hat{𝒜}}_{ℓ} ( h ( x ) , {x}_{ℓ} ) ,$$

where [symbol] is a D-dimensional point, [symbol] (ƒ(x), [symbol] ) is the value measured at [symbol] of an effect that the physical quantity ƒ(x) has on a measuring process [symbol] is a mathematical model of the measuring process [symbol] for the involved [symbol] th measurement, and [symbol] (g(x), [symbol] ) is the value that the mathematical model [symbol] applied to a function g takes at point [symbol] =1, . . . , L.

In a further embodiment of the present invention, the simulations comprise numerical multiphysics simulations or simplified models.

In a further embodiment of the present invention, the specific sets of values, sₙ, comprise a set of sampled parameters s₁, s₂, . . . , s_{P}, p=1, . . . , P, wherein each sampled parameter sₚ varies in a respective rang

$$[ {s}_{p}^{min} , {s}_{p}^{max} ] ,$$

wherein

$${s}_{p}^{min} and {s}_{p}^{max}$$

are fixed bounds, and said specific sets of values, sₙ, are chosen in a space [symbol] given by the Cartesian product of said respective ranges:

$${s}_{n} ∈ 𝒫 = [ {s}_{1}^{min} , {s}_{1}^{max} ] \times [ {s}_{2}^{min} , {s}_{2}^{max} ] \times ⋯ \times [ {s}_{P}^{min} , {s}_{P}^{max} ] .$$

In a further embodiment of the present invention, the set of sampled parameters include random points distributed in the space [symbol] .

In a further embodiment of the present invention, the set of sampled parameters further includes the boundary points given by the Cartesian product of the respective ranges

$$\{ {s}_{p}^{min} , {s}_{p}^{max} \} .$$

In a further embodiment of the present invention, the random points are uniformly distributed in the space [symbol] .

In a further embodiment of the present invention, the system of L equations with N unknowns is solved using the least squares method.

In a further embodiment of the present invention, L<N.

In a further embodiment of the present invention, N/L is in a range from 5 to 50.

In a further embodiment of the present invention, the complex uncertain system is a region of interest of a human body treated by focused deposition of power radiated by an antenna applicator; the physical quantity, ƒ(x), is a temperature T(x) of the treated region of interest; the mathematical model is obtained by using data of a magnetic resonance imaging or a computed tomography of the treated region of interest of said human body; the set of parameters, s, comprises values of dielectric and thermal parameters characterizing different tissues of the region of interest; the scarce set of a number L of indirect measurements, [symbol] (T(x), [symbol] ), is obtained by non-invasive indirect methods, in particular radiometry and microwave tomography, or as a result of temperature measurements obtained through non-invasive intraluminal catheters placed away from the region of interest or by minimally invasive catheters in or near the region of interest.

In a further embodiment of the present invention, the complex uncertain system is an operating electronic device, in particular an integrated circuit or a complex device such as a laptop; the physical quantity, ƒ(x), is a temperature T(x) of the operating device, in particular of its external surface or of one of its internal parts; the mathematical model is obtained by data deriving from drawings or frequency responses of the operating device; the set of parameters, s, comprises a random combination of one or more of electrical, magnetic, chemical, thermal, fluid dynamic and mechanical parameters characterizing the operating device; the scarce set of a number L of indirect measurements, [symbol] (T(x), [symbol] ), is obtained by means of thermal sensors placed in areas of the electronic operating device that are easily accessible or from other indirect measurements.

In a further embodiment of the present invention, the complex uncertain system is a network of communicating agents comprising a plurality of nodes; the physical quantity, ƒ(x), is a spreading process on the network of a pathogen or a malicious software; the mathematical model is obtained from information on relationships between pairs of the plurality of nodes and/or from information on the spatial proximity of the plurality of nodes; the set of parameters, s, comprises reference values of spreading parameters and of a network topology; the scarce set of a number L of indirect measurements is obtained by means of tests performed at the node level or on a plurality of nodes.

The invention will be described in detail hereinafter through non-limiting embodiments with reference to the attached figures, wherein:

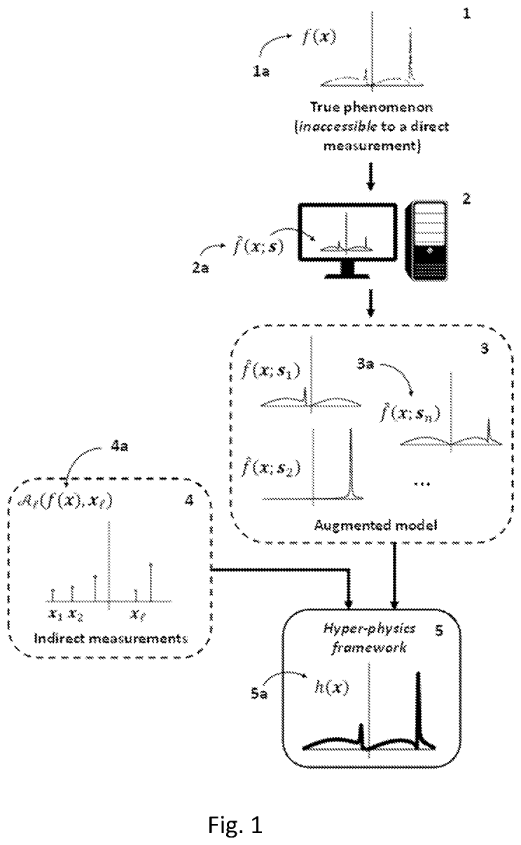

FIG. 1 is a schematic representation of the proposed method, conceived to achieve a reliable assessment of a generic complex phenomenon, of which direct measurements are not available;

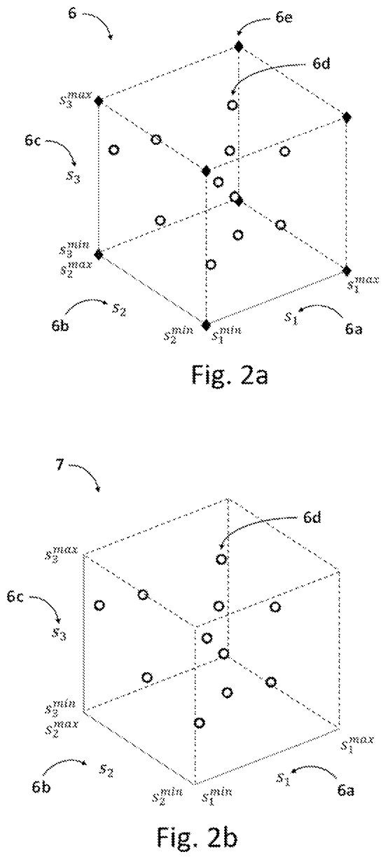

FIGS. 2a and 2b show two respective examples of possible samplings in the space of constituent parameters, when three parameters are considered, wherein each point in the grid corresponds to a member of an augmented model;

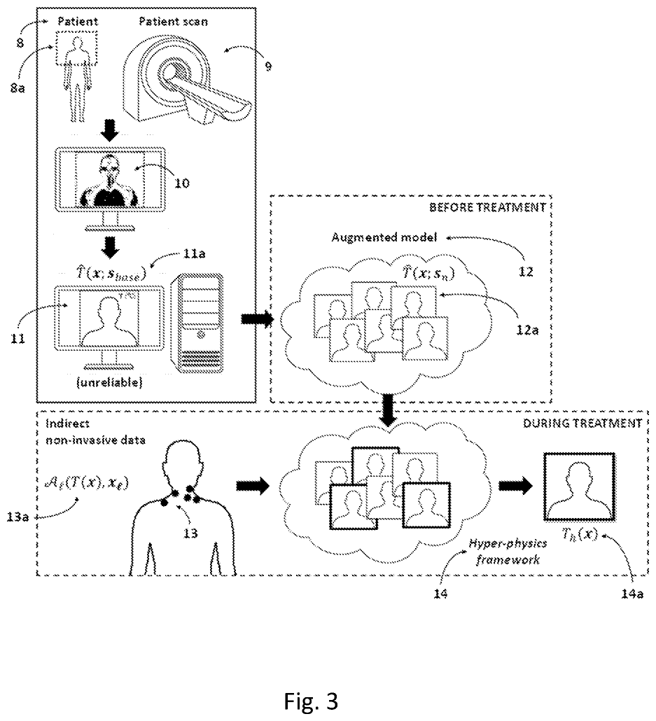

FIG. 3 reports a schematic representation of how the method described in FIG. 1 applies to a real-time construction of reliable temperature maps of a patient in microwave cancer hyperthermia treatments;

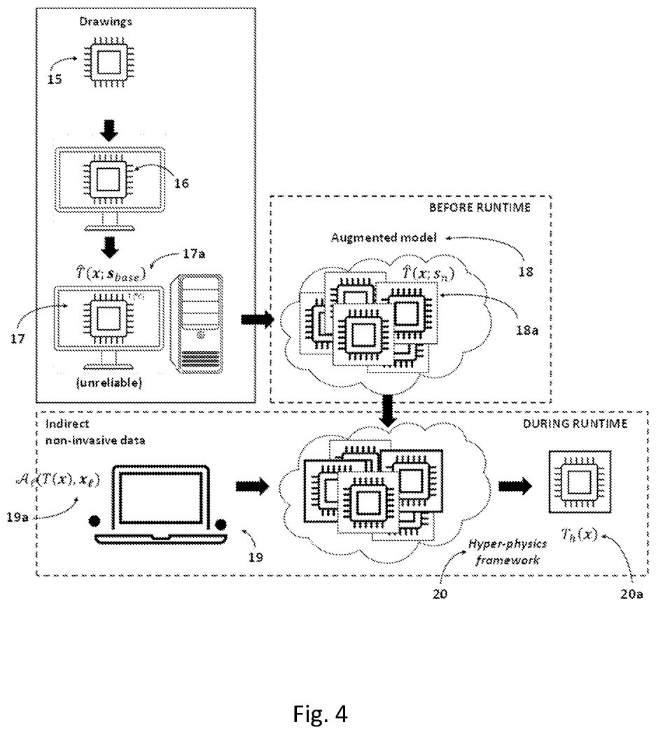

FIG. 4 reports a schematic representation of how the method described in FIG. 1 applies to a real-time temperature monitoring in electronic devices;

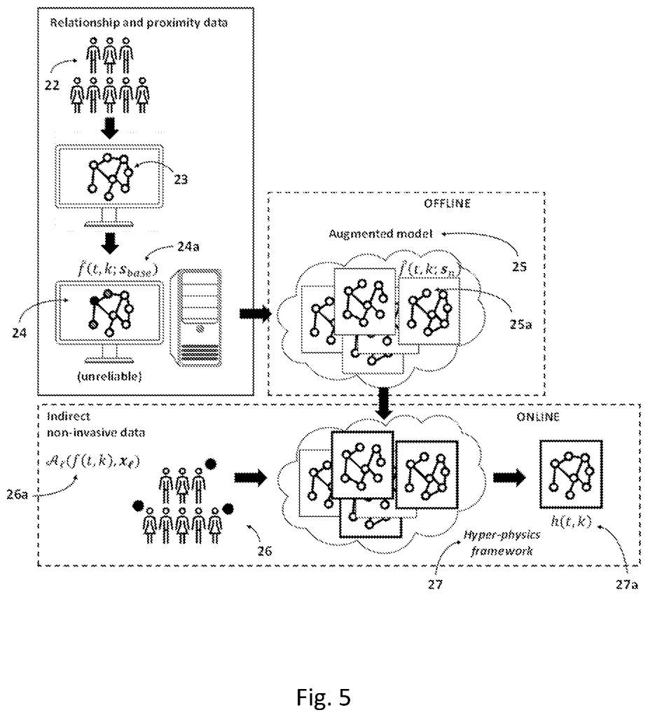

FIG. 5 reports a schematic representation of how the method described in FIG. 1 applies to monitoring a spreading in a network of agents that communicate.

With reference to FIG. 1, a function ƒ: [symbol] → [symbol] ^{Q} is considered, where [symbol] ⊆ [symbol] ^{D}, [symbol] is a field, and the dimension Q is characteristic of the physical process considered; or example, when ƒ is a temperature T, [symbol] = [symbol] and Q=1. The function ƒ describes the distribution in the D-dimensional space ( [symbol] ^{D}) of a physical quantity relative to a true phenomenon 1 characterizing a generic complex uncertain system.

The physical quantity describing the true phenomenon 1, which is assumed inaccessible to direct measurements, is indicated as ƒ(x) (step 1a).

A first step 2 of the procedure consists in describing a case-specific numerical model of the system for simulating the spatial distribution of the physical quantity of interest ƒ(x): an approximated physical quantity obtained through the case-specific numerical model is indicated as \hat{ƒ}(x; s) (step 2a), wherein s is a set of parameters inherent to the model and varying in a space [symbol] ⊆ [symbol] ^{P}, being [symbol] a set and being the dimension P characteristic of the model considered. Then, a library of approximations \hat{ƒ}(x; sₙ), n=1, . . . , N (step 3a), of the function ƒ(x) describing the true phenomenon 1 is obtained with numerical multiphysics simulations or simplified models, i.e., results obtained with physical scaled models of the true complex system, and their possible elaborations.

The distributions \hat{ƒ}(x; sₙ) form the so-called augmented model 3 and are generated by assigning specific sets of values sₙ∈ [symbol] to the constituent parameters describing the system. Once the augmented model 3 is prepared, a key further step of the proposed method consists in looking for an approximation of the physical quantity ƒ(x) related to the true phenomenon 1 in the form:

$$h ( x ) = {\sum }_{n = 1}^{N} {a}_{n} \hat{f} ( x ; {s}_{n} ) , x ϵ 𝒟 , \qquad ( 1 )$$

where aₙ, n=1, . . . , N, are expansion coefficients. The expansion coefficients aₙ, n=1, . . . , N, in equation (1), can be determined by enforcing the condition:

$${\hat{𝒜}}_{ℓ} ( h ( x ) , {x}_{ℓ} ) = {𝒜}_{ℓ} ( f ( x ) , {x}_{ℓ} ) , \qquad ( 2 )$$

where [symbol] is a D-dimensional point, [symbol] (ƒ(x), [symbol] ) is the value measured at [symbol] of the effect that the physical quantity ƒ(x) has on the measuring process [symbol] is a mathematical model of the measuring process for the involved [symbol] th measurement, and [symbol] (g(x), [symbol] ) is the value that the mathematical model [symbol] applied to a function g takes at point [symbol] =1, . . . , L.

When the operators [symbol] are linear—as it is in most practical cases, where the measuring process [symbol] may sample the function at the point [symbol] or can be modelled as a convolution with a known kernel—equation (2) gives rise to a system of L equations with N unknowns. This resulting system can be sold preferably by using the least squares method, to minimize the magnitude of the discrepancies | [symbol] |, where [symbol] =1, . . . , L, and the discrepancies are

$${v}_{ℓ} = {𝒜}_{ℓ} ( f ( x ) , {x}_{ℓ} ) - {\hat{𝒜}}_{ℓ} ( h ( x ) , {x}_{ℓ} ) .$$

In an embodiment, the number of L equations is less than the number N of unknowns.

In a further embodiment, the ratio between the number N of unknowns and the number of L equations is in a range from 5 to 50.

The fundamental goal of the proposed method consists in the possibility to retrieve the most accurate map h(x) (step 5a) of the true phenomenon 1 using a scarce set of indirect measurements [symbol] (ƒ(x), [symbol] ). This is made possible by the a priori information contained in the set of distributions \hat{ƒ}(x; sₙ) obtained at step 3a.

The pre-computed mathematical models of the phenomenon (augmented model 3) and the heterogeneous physically acquired data (indirect measurements 4) are treated in a uniform manner as sources of information and processed concurrently to yield a hyper-physics framework 5 of the system, which provides h(x), that is a reliable and real-time reconstruction, or map, of the physical quantity related to the true phenomenon 1 in the whole domain of interest (step 5a). The information on the entire domain of interest is in fact included in the a priori distributions \hat{ƒ}(x; sₙ) of the augmented model 3, evaluated for xϵ [symbol] .

The diversified nature of the augmented model 3, which takes into account multiple different configurations in which the system may exist, allows a tuning based on imprecise and scarce measurements to orient the reconstruction towards a realistic distribution h(x) of the physical quantity ƒ(x) describing the true phenomenon 1.

As previously mentioned, the augmented model 3 consists of a library of simulations obtained by modeling the system for different combinations sₙ of the constituent parameters s₁, s₂, . . . , s_{P}, whose actual values are unknown.

Assuming each parameter sₚ to vary in a range

$$[ {s}_{p}^{min} , {s}_{p}^{max} ] ,$$

where

$${s}_{p}^{min} and {s}_{p}^{max}$$

are reasonably fixed bounds, the parameters values sₙ can be chosen in the space P given by the Cartesian product of the different ranges, i.e.:

$${s}_{n} ∈ 𝒫 = [ {s}_{1}^{min} , {s}_{1}^{max} ] \times [ {s}_{2}^{min} , {s}_{2}^{max} ] \times ⋯ \times [ {s}_{P}^{min} , {s}_{P}^{max} ] . \qquad ( 3 )$$

Two examples of sampling in the parameters space are reported in FIGS. 2a and 2b, where the case of P=3 constituent parameters s₁ 6a, s₂ 6b and s₃ 6c is considered.

In the example shown in FIG. 2a, the set of sampled parameter values 6 is formed by the boundary points 6e given by the Cartesian product of the discrete sets

$$\{ {s}_{p}^{min} , {s}_{p}^{max} \} , p = 1 , … , P ,$$

and by a certain number of random points 6d distributed in the space defined in equation (3).

With reference to FIG. 2b, the other example of a possible set of sampled parameter values 7 only includes random points 6d distributed in the space defined in equation (3).

The random points 6d may be uniformly distributed in the space defined in equation (3).

The two examples reported in FIG. 2a and FIG. 2b are clearly non-exhaustive, and several alternative samplings can be considered.

A first practical application of the above-described method concerns the problem of the temperature monitoring in microwave hyperthermia treatments, as schematically depicted in FIG. 3. In this case, the complex uncertain system is the human body under thermal stress, and the phenomenon to describe is the temperature increase in the treated region due to the focused deposition of part of the power radiated by the antenna applicator.

With reference to FIG. 3, as usual in hyperthermia pre-treatment planning, patients 8 in treatment position undergo MRI, Magnetic Resonance Imaging, or CT, Computer Tomography, scans 9.

The MRI (or CT) data is then processed in proper tools to perform the patient's specific tissue segmentation of the region of interest (ROI) 8a, producing a patient-specific 3D model 10.

The segmented patient anatomy is then imported in numerical solvers 11 where the baseline values of dielectric and thermal parameters s_{base} (i.e., the values reported in literature) are assigned to the different tissues to generate the temperature map \hat{T}(x; s_{base}) 11a of the patient 8.

Due to the high degree of uncertainty characterizing these parameters, in particular the thermal parameters, the temperature map \hat{T}(x; s_{base}) 11a cannot be considered sufficiently reliable per se. Indeed, the values of parameters s_{base} found in literature for the different tissues could be very different from the actual values, which vary between patients 8, within each tissue, over time, and as non-linear functions of tissue temperature.

The application of the proposed method in this framework implies the generation of a patient-specific augmented model 12, consisting in a set of simulations of purposely mutated replicas \hat{T}(x; sₙ) 12a of the region of interest, where sₙ indicates a random combination of the dielectric and thermal parameters characterizing the different tissues.

By leveraging the inherent information of this set of simulations, the actual temperature distribution in the patient can be conveniently recovered at any time by means of a “model-based interpolation” via a set of indirect measurements 13.

This scarce and inaccurate set of measurements [symbol] (T(x), [symbol] ), [symbol] =1, . . . , L, 13a, is planned to derive from non-invasive indirect methods (e.g., radiometry, microwave tomography), from non-invasive intraluminal catheters placed away from the tumor or region of interest, or from minimally invasive catheters (one or very few).

The resulting hyper-physics framework 14 matches the pre-computed simulative ensemble of states (augmented model 12) to the indirect set of measurements 13a, providing the accurate full temperature map Tₕ(x) 14a of the patient.

This map 14a can be continuously updated over time, providing a real-time monitoring of the patient's temperature during the whole treatment.

A second practical application of the above-described method concerns the problem of the temperature monitoring in electronic devices, as schematically depicted in FIG. 4. In this case, the complex uncertain system is an operating electronic device (e.g., an integrated circuit or a complex device as a laptop), and the phenomenon to be described is the temperature in the device, both on its external surface and in some of its internal parts.

With reference to FIG. 4, data 15 derivable from drawings and other information (e.g., frequency responses) of the operating electronic device with its processing units and with their interconnections are available, as usual in system design.

These data 15 are imported in numerical solvers 16 where the design reference values of dielectric and thermal parameters s_{base} (i.e., the values reported in literature and datasheets) are assigned to the different parts 17 of the operating electronic device to generate the temperature map of the device \hat{T} (x; s_{base}) 17a.

Due to the high degree of uncertainty characterizing these parameters (this uncertainty affects the size and shape of the different parts and their dielectric and thermal parameters, and ambient conditions), the temperature map \hat{T}(x; s_{base}) 17a cannot be considered sufficiently reliable per se. Indeed, the values of the parameters s_{base} could be very different from the actual values due to manufacturing processes.

The application of the proposed method in this framework implies the generation of an augmented model 18 specific to the considered kind of device, consisting in a set of simulations of purposely mutated replicas \hat{T}(x; sₙ) 18a of the device, where sₙ indicates a random combination of one or more of electrical, magnetic, chemical, thermal, fluid dynamic and mechanical parameters characterizing the device.

By leveraging the information contained in this set of simulations, the actual temperature distribution in the device can be conveniently recovered by means of a “model-based interpolation” via a set of indirect measurements 19. This scarce and inaccurate set of measurements [symbol] (T(x), [symbol] ), [symbol] =1, . . . , L 19a, may derive from one or more thermal sensors placed in areas near or on the electronic operating device that are easily accessible, i.e., have the physical conditions compatible with the placement and operation of the sensors, or from other indirect measurements.

The resulting hyper-physics framework 20 matches the pre-computed simulative ensemble of states (augmented model 18) to the indirect set of measurements 19a, providing the accurate full temperature map Tₕ(x) 20a of the device. This map 20a can be continuously updated over time, providing a real-time monitoring of the temperature of the operating electronic device during the complete running time.

A third practical application of the above-described method concerns the problem of monitoring the spreading in a network of agents that communicate. Agents may be people (or communities of people) or computers, for instance. A pathogen, for instance a Coronavirus, or a malicious software may be the quantity spreading over the network. FIG. 5 schematically describes the application of the proposed method to this case of interest. The complex uncertain system is a network of interacting agents, and the phenomenon to be described is the spreading among the nodes of the network.

With reference to FIG. 5, data about the k=1, . . . , K nodes and the links between pairs of them may be available from relationship and proximity data about the agents 22.

These data are imported in numerical solvers 23 where the design reference values of spreading parameters s_{base} (i.e., the values reported in literature) are assigned to the different parts 24 of the network to generate the spreading process on the network \hat{ƒ}(t, k; s_{base}) 24a, with k∈(1, . . . , K) being a vector collecting the indices of the nodes.

Due to the high degree of uncertainty characterizing the model (this uncertainty affects both the spreading parameters and the topology of the network as well), the process \hat{ƒ}(t, k; s_{base}) 24a cannot be considered sufficiently reliable per se. Indeed, the values of the parameters s_{base} could be very different from the actual values.

The application of the proposed method in this framework implies the generation of an augmented model 25, consisting in a set of simulations of purposely mutated replicas of the network \hat{ƒ}(t, k; sₙ) 25a, where sₙ indicates a random combination of the spreading parameters and of the network topology.

By leveraging the information contained in this set of simulations, the actual temperature distribution in the device can be conveniently recovered by means of a “model-based interpolation” via a set of indirect measurements 26. This scarce and inaccurate set of measurements [symbol] (ƒ(t, k), [symbol] ), [symbol] =1, . . . , L, 26a may derive from tests performed at the node level or on a plurality of nodes.

The resulting hyper-physics framework 27 matches the pre-computed simulative ensemble of states (augmented model 25) to the indirect set of measurements 26a, providing the accurate full evolution map h(t, k) 27a of the spreading over the network. This map 27a can be continuously updated over time, providing a real-time monitoring of the spreading over the network.

The advantages of the present invention are therefore evident from the description provided above.

The computer implemented method for obtaining real-time maps of physical quantities characterizing complex uncertain systems from scarce and indirect measurements advantageously provide real-time complete, accurate, physically sound, and reliable maps of the quantity of interest.

Moreover, the computer implemented method according to the invention can advantageously be applied to any uncertain system.

Further, in the computer implemented method according to the present invention the indirect measurement data can be obtained by means of non-invasive techniques. This is particular advantageous when the method is applied in medical treatments related to the human body.

The present description has tackled some of the possible variants, but it will be apparent to the man skilled in the art that other embodiments may also be implemented, wherein some elements may be replaced with other technically equivalent elements. The present invention is not therefore limited to the explanatory examples described herein, but may be subject to many modifications, improvements or replacements of equivalent parts and elements without departing from the basic inventive idea, as set out in the following claims.

1. A computer implemented method for obtaining a real-time map, h(x), of a distribution in a D-dimensional space of a physical quantity, ƒ(x), describing a true phenomenon characterizing a complex uncertain system, wherein said physical quantity, ƒ(x), is inaccessible to direct measurements, said method comprising the steps of:
describing said complex uncertain system by means of a mathematical model for simulating a spatial distribution, \hat{ƒ}(x; s), of said physical quantity, ƒ(x), using a set of parameters (s);
building a library of approximations, \hat{ƒ}(x; sₙ), n=1, . . . , N, of said physical quantity, ƒ(x), by running a plurality of simulations through assignment of specific sets of values, sₙ, to said set of parameters, s;
calculating said real-time map, h(x), representing a real-time approximation of said physical quantity, ƒ(x), in a form
$$h ( x ) = {\sum }_{n = 1}^{N} {a}_{n} \hat{f} ( x ; {s}_{n} ) ,$$
wherein aₙ, n=1, . . . , N, are expansion coefficients which are determined by solving a system of L equations with N unknowns so as to minimize the magnitude of the discrepancies | [symbol] |, where [symbol] =1, . . . , L, and the discrepancies are
$${v}_{ℓ} = {𝒜}_{ℓ} ( f ( x ) , {x}_{ℓ} ) - {\hat{𝒜}}_{ℓ} ( h ( x ) , {x}_{ℓ} ) ,$$
where [symbol] is a D-dimensional point, [symbol] (ƒ(x), [symbol] ) is the value measured at xₗ of an effect that the physical quantity ƒ(x) has on a measuring process, [symbol] is a mathematical model of the measuring process [symbol] for the involved [symbol] th measurement, and [symbol] (g(x), [symbol] ) is the value that the mathematical model [symbol] applied to a function g takes at point [symbol] =1, . . . , L.

2. The computer implemented method according to claim 1, wherein said simulations comprise numerical multiphysics simulations or simplified models.

3. The computer implemented method according to claim 1, wherein said specific sets of values, sₙ, comprise a set of sampled parameters s₁, s₂, . . . , s_{P}, p=1, . . . , P, wherein each sampled parameter sₚ varies in a respective range
$$[ {s}_{p}^{min} , {s}_{p}^{max} ] ,$$
wherein
$${s}_{p}^{min} and {s}_{p}^{max}$$
are fixed bounds, and said specific sets of values, sₙ, are chosen in a space [symbol] given by the Cartesian product of said respective ranges:
$${s}_{n} ∈ 𝒫 = [ {s}_{1}^{min} , {s}_{1}^{max} ] \times [ {s}_{2}^{min} , {s}_{2}^{max} ] \times ⋯ \times [ {s}_{P}^{min} , {s}_{P}^{max} ] .$$

4. The computer implemented method according to claim 3, wherein said set of sampled parameters include random points distributed in said space [symbol] .

5. The computer implemented method according to claim 4, wherein said set of sampled parameters further includes the boundary points given by the Cartesian product of said respective ranges
$$\{ {s}_{p}^{min} , {s}_{p}^{max} \} .$$

6. The computer implemented method according to claim 4, wherein said random points are uniformly distributed in said space [symbol] .

7. The computer implemented method according to claim 1, wherein said system of L equations with N unknowns is solved using the least squares method.

8. The computer implemented method according to claim 1, wherein L<N.

9. The computer implemented method according to claim 1, wherein N/L is in a range from 5 to 50.

10. The computer implemented method according to claim 1, wherein:
said complex uncertain system is a region of interest of a human body treated by focused deposition of power radiated by an antenna applicator;
said physical quantity, ƒ(x), is a temperature T(x) of said treated region of interest;
said mathematical model is obtained by using data of a magnetic resonance imaging or a computed tomography of said treated region of interest of said human body;
said set of parameters, s, comprises values of dielectric and thermal parameters characterizing different tissues of said region of interest;
said scarce set of a number L of indirect measurements, [symbol] (T(x), [symbol] ), is obtained by non-invasive indirect methods, in particular radiometry and microwave tomography, or as a result of temperature measurements obtained through non-invasive intraluminal catheters placed away from the region of interest or by minimally invasive catheters in or near the region of interest.

11. The computer implemented method according to claim 1, wherein:
said complex uncertain system is an operating electronic device, in particular an integrated circuit or a complex device such as a laptop;
said physical quantity, ƒ(x), is a temperature T(x) of said operating device, in particular its external surface or one of its internal parts;
said mathematical model is obtained by data deriving from drawings or frequency responses of said operating device;
said set of parameters, s, comprises a random combination of one or more of electrical, magnetic, chemical, thermal, fluid dynamic and mechanical parameters characterizing said operating device;
said scarce set of a number L of indirect measurements, [symbol] (T(x), [symbol] ), is obtained by means of thermal sensors placed in areas of the electronic operating device that are easily accessible or from other indirect measurements.

12. The computer implemented method according to claim 1, wherein:
said complex uncertain system is a network of communicating agents comprising a plurality of nodes;
said physical quantity, ƒ(x), is a spreading process on the network of a pathogen or a malicious software;
said mathematical model is obtained from information on relationships between pairs of said plurality of nodes and/or from information on the spatial proximity of said plurality of nodes;
said set of parameters, s, comprises reference values of spreading parameters and of a network topology;
said scarce set of a number L of indirect measurements is obtained by means of tests performed at the node level or on a plurality of nodes.
